(19) United States
(12) Patent Application Publication
(10) Pub. No.: US 2011/0313046 A1
(43) Pub. Date: Dec. 22, 2011

(54) COMBINATION THERAPY WITH LISDEXAMPHETAMINE AND EXTENDED RELEASE GUANFACINE

(76) Inventor: James ERMER, Eagleville, PA (US)

(21) Appl. No.: 13/157,849

(22) Filed: Jun. 10, 2011

Related U.S. Application Data
(60) Provisional application No. 61/353,858, filed on Jun. 11, 2010.

(51) Int. Cl. A61K 31/165; A61P 25/00

(52) U.S. Cl. 514/617

(57) ABSTRACT

The present invention relates to treating children and adults with suboptimal responses to Attention Deficit Hyperactivity Disorder (ADHD) monotherapy. More specifically, the present invention relates to a method for treating ADHD in a patient which comprises administering to the patient an extended release guanfacine composition adjunctively with a lisdexamphetamine composition.

This application claims priority to U.S. Provisional Application No. 61/353,858, filed Jun. 11, 2010, the contents of which are hereby incorporated by reference in their entirety.

The present invention relates to treating children and adults with suboptimal responses to Attention Deficit Hyperactivity Disorder (ADHD) monotherapy. More specifically, the present invention relates to a method for treating ADHD in a patient which comprises administering to the patient an extended release guanfacine composition adjunctively with a lisdexamphetamine composition.

### BACKGROUND

Attention-deficit hyperactivity disorder (ADHD) is a heterogeneous neurobehavioral disorder characterized by a persistent pattern of developmentally inappropriate inattentiveness, impulsivity, and hyperactivity. It often occurs in the presence of other comorbid conditions. The diagnosis of ADHD is made by a healthcare professional who applies either International Statistical Classification of Diseases and Related Health Problems 10th Revision (ICD-10®) or Diagnostic and Statistical Manual of Mental Disorders, 4th ed.-Text Revision (DSM-IV-TR®) (American Psychiatric Association 2000) criteria. Symptoms of ADHD lasting at least 6 months must be shown to interfere with age-appropriate functioning in at least 2 settings (eg, social, academic, or occupational) that cannot be accounted for by another psychiatric disorder. According to the DSM-IV-TR criteria, there are 3 ADHD sub-types: hyperactive/impulsive, inattentive, or combined type. Depending on the ADHD subtype, gender, and the presence of comorbid disorders, individuals with ADHD may differ considerably, even within a particular age cohort.

ADHD is one of the most common neurodevelopmental disorders of childhood, and consequently, prevalence rates have been extensively investigated. Literature-reported rates vary; however, the preponderance of variance is thought to be due to methodological differences. The worldwide prevalence of ADHD in children 18 years of age or younger can be estimated to be 5.3% when adjusted for methodological differences. No significant differences in ADHD prevalence rates between North America, Europe, and other parts of the world were detected in this meta-regression analysis involving 102 studies and 171,756 subjects (Polanczyk et al., Am J. Psych. 2007; 164(6):942-48).

The exact etiology of ADHD is unknown. Extensive research has suggested that neurotransmitter deficits (Arnsten et al., Arch Gen Psych. 2001; 53(5):448-55), genetics (Arnsten 2001; Brown K, Neuroscience, 2003; 301(5630):160-1), environment (Kahn et al., J. Pediatr. 2003; 143(1):104-10), and perinatal complications (Bhutta et al., JAMA 2002; 288(6):728-37) may all be contributing factors. It has been hypothesized that the mechanism of action of effective medications in ADHD is to raise the levels of neurotransmitters (specifically norepinephrine and/or dopamine, or their precursors) at the synapse either by facilitating their release, decreasing their reuptake, or binding and activating the post-synaptic receptor (Kratochvil et al., Expert Opin Pharmacother. 2003; 4(7):1165-74; Wang et al., Cell 2007; 129(2):397-410).

Psychostimulant medications, such as methylphenidate and amphetamine, have been used to treat behavior problems in children since 1937 (Wilens and Biederman, Psychiatr Clin North Am. 1992; 15(1):191-222), including ADHD and its diagnostic precursors. The Multimodal Treatment Study of Children with ADHD Cooperative Group showed that, for intermediate to long-term treatment (14 months) of ADHD, psychostimulant treatment (methylphenidate HCl) titrated to effect followed by monthly visits, was superior to behavioral treatment alone and to routine community care that included medication (The MTA Cooperative Group, Arch Gen Psych. 1999; 56(12):1073-86).

Despite the effectiveness of psychostimulant medications, some patients have a suboptimal response (Olfson, Am J Manag Care, 2004; 10 (suppl 4):S117-24) or have side effects and possible exacerbation of some common co-morbid conditions (e.g., anorexia, tics, and insomnia) that limit their ability to reach an optimal dose.

Guanfacine is a selective α₂ agonist that is approved for the treatment of ADHD. Based on preclinical studies, the mechanism of action of guanfacine is hypothesized to be due to an effect on the dorsolateral prefrontal cortex (DLPFC), where guanfacine is thought to enhance the effect of norepinephrine at post-synaptic α_{2A}-adrenoreceptors (Arnsten et al., Arch Gen Psych. 1996; 53(5):448-55) and increase regional cerebral blood flow (Avery et al., Neuropsychopharmacol. 2000; 23(3):240-9) to improve the cognition and behavior in patients with ADHD; however, the exact mechanism of action is unknown. The effects on DLPFC seen in animals were also demonstrated in a recent functional magnet resonance imaging study in normal adult humans who were scanned at Baseline and following 1 mg of immediate-release guanfacine administration (Clerkin et al. Biol Psych. 2009; 66(4):307-12). It has also been shown that the delayed response performance in young intact rhesus monkeys can be improved by guanfacine (Franowicz and Arnsten, Psychopharmacol. (Berl) 2002; 162(3):304-12).

An assessment of immediate-release guanfacine prescriptions revealed that 42% of the prescriptions for immediate-release guanfacine were also prescribed with a psychostimulant (IMS 2009). Extended-release guanfacine HCl, was approved as INTUNIV® in September 2009.

### SUMMARY OF THE INVENTION

The present invention addresses the problem of treating children and adults with suboptimal responses to ADHD monotherapy. More specifically, the present invention includes a method for treating Attention Deficit Hyperactivity Disorder (ADHD) in a patient which comprises administering to the patient an extended release guanfacine composition adjunctively with a lisdexamphetamine composition.

In an aspect of the invention, the extended release guanfacine composition is administered orally once per day. Further, the extended release guanfacine composition may be administered in the morning or in the evening. According to the invention, the extended release guanfacine composition dosage may be 1 mg, 2 mg, 3 mg, or 4 mg. Further, in an embodiment of the invention, the present invention provides that the extended release guanfacine composition dosage may be 0.05 mg/kg/day to 0.12 mg/kg/day. In another embodiment, the present invention provides that the extended release guanfacine composition dosage may be 0.05 mg/kg/day to 0.14 mg/kg/day

In an embodiment of the present invention, the lisdexamphetamine composition comprises lisdexamphetamine dimesylate. In a further embodiment, the lisdexamphetamine composition comprises lisdexamfetamine dimesylate, microcrystalline cellulose, croscarmellose sodium, and magnesium stearate. According to the present invention, the lisdexamphetamine may be administered in a single daily dose of 20 mg, 30 mg, 40 mg, 50 mg, 60 mg, or 70 mg.

The present invention provides that the extended release guanfacine composition may comprise guanfacine hydrochloride, hypromellose and methacrylic acid copolymer.

In an embodiment of the invention, the patient is a child. In another embodiment, the patient is an adolescent. In a further embodiment, the patient is an adult.

### BRIEF DESCRIPTION OF THE FIGURES

FIG. 1 is a graph showing the mean guanfacine concentrations over time following administration of extended release guanfacine (SPD503, INTUNIV®) alone and in combination with lisdexamphetamine (VYVANSE®).

FIG. 2 is a graph showing the mean d-amphetamine concentrations over time following administration of VYVANSE® alone and in combination with SPD503.

FIG. 3 is a graph showing the mean lisdexamphetamine concentrations over time following administration of lisdexamphetamine alone and in combination with extended release guanfacine.

### DETAILED DESCRIPTION

Pharmacokinetic drug-drug interactions can occur when two drugs are co-administered, resulting in a change in the metabolism, absorption, tissue and/or plasma binding, distribution, or elimination of one or both drugs. Guanfacine is known to be metabolized by CYP3A4. Lisdexamphetamine is not metabolized by the CYP450 system, and is neither an inducer nor inhibitor of the system.

### DEFINITIONS

An “adverse event” (AE) is any untoward medical occurrence in a clinical investigation subject administered a pharmaceutical product and which did not necessarily have a causal relationship with the treatment.

A “treatment-emergent adverse event” (TEAE) is an AE that occurred or worsened during the on-treatment period.

A “non-treatment-emergent adverse event” is an AE that occurred during the pre-treatment period or the post-treatment period.

“Cₘₐₓ” is the maximum plasma concentration.

“Tₘₐₓ” is the time to Cₘₐₓ.

“AUC_{0-∞}” is the area under the plasma concentration versus time curve extrapolated to infinity.

“T_{1/2}” is the apparent terminal half-life.

“CL/F” is the apparent oral-dose clearance.

“Vz/F” is the apparent volume of distribution.

Extended Release Guanfacine:

Guanfacine hydrochloride is a 2-adrenoceptor agonist. The chemical name of guanfacine hydrochloride is N-Amidino-2-(2,6-dichlorophenyl) acetamide monohydrochloride and its molecular weight is 282.55.

The chemical structure of guanfacine hydrochloride is:

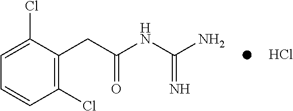

An extended release guanfacine formulation according to the present invention preferably comprises guanfacine hydrochloride, hypromellose and methacrylic acid copolymer. The extended release guanfacine administered in the study described in the Example was an extended release guanfacine hydrochloride (INTUNIV®) provided by Shire Pharmaceuticals.

Lisdexamphetamine:

A lisdexamphetamine formulation according to the present invention preferably comprises lisdexamfetamine dimesylate, microcrystalline cellulose, croscarmellose sodium, and magnesium stearate. The lisdexamphetamine administered in the study described in the Example was VYVANSE® (Shire Pharmaceuticals). VYVANSE® is lisdexamphetamine dimesylate.

The chemical structure of lisdexamphetamine is:

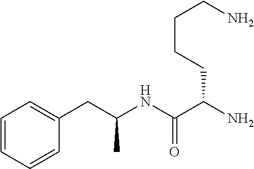

Dosage and Administration:

The preferred route of administration for the lisdexamphetamine and extended release guanfacine of the present invention is oral. Other routes of administration include rectal, sublingual, and any other transmucosal route. The lisdexamphetamine and extended release guanfacine can be administered as separate dosage forms, e.g., tablets or capsules. According to the present invention, the lisdexamphetamine and extended release guanfacine can be administered together in a single dosage unit, e.g., a tablet containing lisdexamphetamine and extended release guanfacine. In combination therapy, the dosage and frequency of administration of each active ingredient of the combination can be controlled independently. For example, one active ingredient may be administered three times per day, while the second compound may be administered once per day. The compounds may also be formulated together such that one administration delivers both compounds.

A dosage unit containing extended release guanfacine and lisdexamphetamine according to the present invention can be in the form of any conventional form for including two active agents, e.g., a bi-layer tablet, a solid dosage form containing a matrix, a capsule containing populations of beads.

The dosage amount of extended release guanfacine can be 0.5 mg to 10 mg. In certain embodiments, the dosage amount of the extended release guanfacine is 0.5 mg, 1 mg, 2 mg, 3 mg, 4 mg, 5 mg, 6 mg, 7 mg, 8 mg, 9 mg, or 10 mg. The extended release guanfacine dosage may be administered in one daily dose or in divided doses. In an embodiment of the invention, the daily dosage amount is 4 mg. In a further embodiment, the daily dosage is 0.05 mg/kg to 0.08 mg/kg or 0.09-0.12 mg/kg.

The dosage amount of lisdexamphetamine can be 1 mg to 100 mg. In certain embodiments, the dosage amount of lisdexamphetamine is 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. The lisdexamphetamine dosage may be administered in one dose or in divided doses. Thus, for example, lisdexamphetamine may be administered in a single daily dose of 20 mg, 30 mg, 40 mg, 50 mg, 60 mg, or 70 mg.

Thus, in the case of a unit dosage form containing extended release guanfacine and lisdexamphetamine, the dosage amounts can be, for example, 20 mg of lisdexamphetamine and 2 mg of extended release guanfacine. Any combination of a suitable dosage amount of each of the active ingredients (lisdexamphetamine and extended release guanfacine) is suitable according to the instant invention.

The dosage of each compound of the claimed combinations depends on several factors, including: the administration method, the disease to be treated, the severity of the disease, whether the disease is to be treated or prevented, and the age, weight, and health of the person to be treated.

In an aspect of the invention, extended release guanfacine is added to the patient's existing lisdexamphetamine treatment for ADHD. Thus, for example, a patient with a suboptimal response to lisdexamphetamine is started on 1 mg/day of extended release guanfacine and titrated up to a maximum of 4 mg/day based on tolerability and response. In this example, the patient is maintained on the maximum dosage.

In one aspect of the invention, the combination of lisdexamphetamine and extended release guanfacine is administered to a patient to treat attention deficit hyperactivity disorder (ADHD). The patient can be, for example, an adult human, a human adolescent (age 13-17) ora human child (e.g., age 6-12 years). In an embodiment of the invention, the combination of lisdexamphetamine and extended release guanfacine can be administered to treat a human with ADHD who was non-responsive or partially-responsive to single-agent treatment. In another embodiment, the combination of lisdexamphetamine and extended release guanfacine can be administered to treat a human who suffered from side effects from other ADHD therapy. In a further embodiment, the combination of lisdexamphetamine and extended release guanfacine can be administered to a human as first-line treatment for ADHD.

Pharmaceutical Compositions

Pharmaceutical compositions of the present invention may include any appropriate amount of the active ingredients in any suitable pharmaceutical carrier substance. The composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each active ingredient in the combination may be formulated in a variety of ways that are known in the art. For example, the ingredients may be formulated together or separately. Thus, the ingredients may be formulated together for the simultaneous or near simultaneous administration of the agents. Such co-formulated compositions can include lisdexamphetamine and extended release guanfacine formulated together in the same pill, capsule, etc. By using different formulation strategies for different agents, the pharmacokinetic profiles for each agent can be suitably matched.

The individually or separately formulated ingredients can be packaged together as a kit. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Dosage forms can be made according to well known methods in the art. Some preferred methods are described below.

Bilayer Tablets

A bilayer tablet can be formulated for a combination of the invention in which different custom granulations are made for each active ingredient of the combination and the two active ingredients are compressed on a bi-layer press to form a single tablet.

Matrix Dosage Forms

The term matrix, as used herein, is given its well known meaning in the pharmaceutical arts, that is a solid material having an active agent incorporated therein. Upon exposure to a dissolution media, channels are formed in the solid material so that the active agent can escape. Dosage forms according to one embodiment of the present invention may be in the form of coated or uncoated matrices. A coating, for example may contain lisdexamphetamine alone and the matrix itself can contain, for example, extended release guanfacine alone or in combination with lisdexamphetamine.

The skilled artisan will appreciate that the matrix material can be chosen from a wide variety of materials which can provide the desired dissolution profiles. Materials can include, for example, one or more gel forming polymers such as polyvinyl alcohol, cellulose ethers including, for example, hydroxy propyl alkyl, celluloses such as hydroxypropyl methyl cellulose, hydroxy alkyl celluloses such as hydroxy propyl cellulose, natural or synthetic gums such as guar gum, xanthum gum, and alginates, as well as, ethyl cellulose, polyvinyl pyrrolidone, fats, waxes, polycarboxylic acids or esters such as the Carbopol®. (Noveon IP Holdings, Corporation) series of polymers, methacrylic acid copolymers, and methacrylate polymers.

Methods of making matrix dosages are well known in the art and any known method of making such dosages which yields the desired release dissolution profiles can be used. One such method involves the mixture of the active ingredient combination with a solid polymeric material and one or more pharmaceutically acceptable excipients which are then blended and compressed in controlled release tablet cores. Such tablet cores can be used for further processing as bi-layer tablets, press coated tablets, or film coated tablets.

A coating can be added to the outside of the tablet core to produce a final dosage form. Such a coating can be spray coated onto the tablet cores. The coating may also be applied using a press-coating process. Press coating techniques are known in the art and are described in U.S. Pat. No. 6,372,254 to Ting et al., incorporated herein by reference in its entirety.

In addition, the formulation of release components can occur by appropriate granulation methods as is well known in the art. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets.

Particle Based Dosage Forms

The immediate release/controlled release dosage forms of the present invention can also take the form of pharmaceutical particles. The dosage forms can include immediate release particles in combination with controlled release particles in a ratio sufficient to deliver the desired dosages of active ingredients. The controlled release particles can be produced by coating the immediate release particles.

The particles can be produced according to any of a number of well known methods for making particles. The immediate release particles comprise the active agent combination and a disintegrant. Suitable disintegrants include, for example, starch, low-substitution hydroxypropyl cellulose, croscarmellose sodium, calcium carboxymethyl cellulose, hydroxypropyl starch, and microcrystalline cellulose.

In addition to the above-mentioned ingredients, a controlled release matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A controlled release matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

Particles can assume any standard structure known in the pharmaceutical arts. Such structures include, for example, matrix particles, non-pareil cores having a drug layer and active or inactive cores having multiple layers thereon. A controlled release coating can be added to any of these structures to create a controlled release particle.

The term particle as used herein means a granule having a diameter of between about 0.01 mm and about 5.0 mm, preferably between about 0.1 mm and about 2.5 mm, and more preferably between about 0.5 mm and about 2 mm. The skilled artisan will appreciate that particles according to the present invention can be any geometrical shape within this size range and so long as the mean for a statistical distribution of particles falls within the particle sizes enumerated above, they will be considered to fall within the contemplated scope of the present invention.

The release of the therapeutically active ingredient from the controlled release formulation of the present invention can be further influenced, i.e., adjusted to a desired rate, by the addition of one of more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

In one embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another embodiment, a suitable amount of the particles are compressed into an oral tablet using conventional tableting equipment using standard techniques. Techniques and compositions for making tablets (compressed and molded), capsules (hard and soft gelatin), and pills are also described in Remington's Pharmaceutical Sciences, Arthur Osol, editor, pp. 1553 1593 (1980), incorporated herein by reference. The particles can be made by mixing the relevant ingredients and granulating the mixture. The resulting particles are dried and screened, and the particles having the desired size are used for drug formulation.

Controlled Release Particles

The controlled release particles of the present invention slowly release the active ingredients when ingested and exposed to gastric fluid and intestinal fluids. The controlled release profile of the formulations of the invention can be altered, for example, by increasing or decreasing the thickness of the retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material may include an effective amount of plasticizer, e.g. triethyl citrate. Preformulated aqueous dispersions of ethylcellulose, such as Aquacoat® (FMC Corporation) or Surelease® (Colorcon, Inc., West Point, Pa., U.S.A), may be used.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylicacid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methycellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS (Rohm Pharma) lacquer solutions include polyethylene glycols, propylene glycol, diethyl phthalate, castor oil, and triacetin. Triethyl citrate is a preferred plasticizer for aqueous dispersions of ethyl cellulose.

One commercially available aqueous dispersion of ethylcellulose is Aquacoat® (FMC Corporation) which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease®(Colorcon, Inc., West Point, Pa., U.S.A.).

In one embodiment, the acrylic coating is an acrylic resin lacquer used in the form of an aqueous dispersion, such as that which is commercially available from Rohm Pharma under the trade name Eudragit®. In additional embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 are copolymers of acrylic and methacrylic esters with a low content of quaternary ammonium groups, the molar ratio of ammonium groups to the remaining neutral (meth)acrylic esters being 1:20 in Eudragit® RL 30 and 1:40 in Eudragit® RS 30 D. The mean molecular weight is about 150,000 Daltons. Eudragit® RL/RS(Rohm Pharma) mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a controlled-release formulation having a desirable dissolution profile. Of course, one skilled in the art will recognize that other acrylic polymers may also be used. In addition to modifying the dissolution profile by altering the relative amounts of different acrylic resin lacquers, the dissolution profile of the ultimate product may also be modified, for example, by increasing or decreasing the thickness of the retardant coating.

Spheroids or beads coated with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product which includes hydroxypropyl methycellulose with or without colorant (e.g., Opadry®, commercially available from Colorcon, Inc.) may be added to the solution and the solution mixed (e.g., for about 1 hour) prior to application onto the beads. The resultant coated substrate, beads in this example, may then be optionally overcoated with a barrier agent to separate the therapeutically active agent from the hydrophobic controlled release coating. An example of a suitable barrier agent is one which comprises hydroxypropylmethylcellulose. However, any film-former known in the art may be used. It is preferred that the barrier agent does not affect the dissolution rate of the final product.

Immediate release particles according to the present invention may be coated with a controlled release coating in order to change the release rate to obtain the dissolution rates according to the present invention.

Press Coated, Pulsatile Dosage Form

In another embodiment of the present invention, the active ingredient combination is administered via a press coated pulsatile drug delivery system suitable for oral administration with a controlled release component, which contains a compressed blend of an active ingredient and one or more polymers, substantially enveloped by an immediate release component, which contains a compressed blend of the active agent and hydrophilic and hydrophobic polymers. The immediate-release component preferably comprises a compressed blend of active agent and one or more polymers with disintegration characteristics such that the polymers disintegrate rapidly upon exposure to the aqueous medium.

The controlled-release component preferably comprises a combination of hydrophilic and hydrophobic polymers. In this embodiment, once administered, the hydrophilic polymer dissolves away to weaken the structure of the controlled-release component, and the hydrophobic polymer retards the water penetration and helps to maintain the shape of the drug delivery system.

In accordance with the present invention, the term “polymer” includes single or multiple polymeric substances, which can swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, and zein, and combinations thereof.

The term “hydrophilic polymers” as used herein includes, for example, one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethycellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The hydrophobic polymer of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

The present invention also provides a method for preparing a press coated, pulsatile drug delivery system suitable for oral administration. This method includes the steps of combining an effective amount of an active ingredient, or a pharmaceutically acceptable salt thereof, and a polymer to form an immediate-release component; combining an effective amount of an active agent, or a pharmaceutically acceptable salt thereof, and a combination of hydrophilic and hydrophobic polymers to form an controlled-release component; and press coating the controlled-release component to substantially envelop the immediate-release component.

An embodiment further includes the steps of combining an effective amount of an active ingredient, or a pharmaceutically acceptable salt thereof, and a polymer to form an immediate-release component, and press coating the immediate-release component to substantially envelop the controlled-release component. In another embodiment, the combining steps can be done by blending, wet granulation, fluid-bed granulation, or dry granulation according to methods recognized in the art.

Liposomal Formulations

One or both active ingredients of the combinations of the invention, can be incorporated into liposomal carriers for administration. The liposomal carriers are composed of three general types of vesicle-forming lipid components. The first includes vesicle-forming lipids that will form the bulk of the vesicle structure in the liposome. Generally, these vesicle-forming lipids include any amphipathic lipids having hydrophobic and polar head group moieties, and which (a) can form spontaneously into bilayer vesicles in water, as exemplified by phospholipids, or (b) are stably incorporated into lipid bilayers, with its hydrophobic moiety in contact with the interior, hydrophobic region of the bilayer membrane, and its polar head group moiety oriented toward the exterior, polar surface of the membrane.

The vesicle-forming lipids of this type are preferably ones having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. The above-described lipids and phospholipids whose acyl chains have a variety of degrees of saturation can be obtained commercially, or prepared according to published methods. Other lipids that can be included in the invention are glycolipids and sterols, such as cholesterol.

The second general component includes a vesicle-forming lipid that is derivatized with a polymer chain that will form the polymer layer in the composition. The vesicle-forming lipids that can be used as the second general vesicle-forming lipid component are any of those described for the first general vesicle-forming lipid component. Vesicle forming lipids with diacyl chains, such as phospholipids, are preferred. One exemplary phospholipid is phosphatidylethanolamine (PE), which provides a reactive amino group that is convenient for coupling to the activated polymers. An exemplary PE is distearyl PE (DSPE).

A suitable polymer is the derivatized lipid polyethyleneglycol (PEG), particularly a PEG chain having a molecular weight between 1,000-15,000 daltons, more particularly between 2,000 and 10,000 daltons, most particularly between 2,000 and 5,000 daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Additionally, block copolymers or random copolymers of these polymers, particularly including PEG segments, may be suitable. Methods for preparing lipids derivatized with hydrophilic polymers, such as PEG, are well known e.g., as described in U.S. Pat. No. 5,013,556.

A third general vesicle-forming lipid component, which is optional, is a lipid anchor by which a targeting moiety is anchored to the liposome, through a polymer chain in the anchor. Additionally, the targeting group is positioned at the distal end of the polymer chain in such a way so that the biological activity of the targeting moiety is not lost. The lipid anchor has a hydrophobic moiety which serves to anchor the lipid in the outer layer of the liposome bilayer surface, a polar head group to which the interior end of the polymer is covalently attached, and a free (exterior) polymer end which is or can be activated for covalent coupling to the targeting moiety.

The lipid components used in forming the liposomes may be present in a molar ratio of about 70-90 percent vesicle forming lipids, 1-25 percent polymer derivatized lipid, and 0.1-5 percent lipid anchor. One exemplary formulation includes 50-70 mole percent underivatized PE, 20-40 mole percent cholesterol, 0.1-1 mole percent of a PE-PEG (3500) polymer with a chemically reactive group at its free end for coupling to a targeting moiety, 5-10 mole percent PE derivatized with PEG 3500 polymer chains, and 1 mole percent alpha-tocopherol.

The liposomal formulations of the present invention include at least one surface-active agent. Suitable surface-active agents useful for the formulation of the combinations described herein include compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants.

### Example 1

An open-label, randomized, single-center, 3-period, crossover, drug-drug interaction study in 42 healthy adults (18-45 years old) was performed. Subjects received three single, oral dose treatments: 4 mg extended release guanfacine, 50 mg lisdexamphetmaine, and 4 mg extended release guanfacine in combination with 50 mg lisdexamphetmaine. Subjects underwent the three treatment periods with a 7-day washout between each treatment.

Pharmacokinetic Results:

The 90% confidence interval (CI) of the geometric mean ratio of guanfacine following combination therapy with extended release guanfacine and lisdexamphetamine following extended release guanfacine alone fell within the standard interval of bioequivalence of 0.80 to 1.25 for AUC₀₋ₜ, and AUC_{0-α}. The 90% CI of the geometric mean ratio of guanfacine Cₘₐₓ following extended release guanfacine in combination with lisdexamphetamine to guanfacine following extended release guanfacine alone exceeded the upper bound of the standard interval of bioequivalence by 7%.

The guanfacine Cₘₐₓ was increased by 19% when co-administered with lisdexamphetamine.

The 90% CI of the geometric mean ratio of d-amphetamine following lisdexamphetamine in combination with extended release guanfacine to d-amphetamine following lisdexamphetamine alone fell within the interval (0.80, 1.25) for Cₘₐₓ, AUC₀₋ₜ, and AUC_{0-∞}.

Forty-three percent of subjects reported at least one treatment-emergent adverse event (TEAE). None of the TEAEs were unexpected. The most frequently reported TEAE was dizziness. There were no differences in type, incidence, or severity of TEAE between treatment regimens.

No serious adverse events (SAEs) were reported. There were no clinically meaningful changes to electrocardiograms (ECGs), clinical laboratory parameters, or physical examinations during the study. The overall effect on pulse rate, blood pressure, and orthostatic vital sign changes was consistent with what has previously been observed with each drug given alone.

Guanfacine Pharmacokinetic Results:

A summary of guanfacine plasma concentrations following the administration of extended release guanfacine alone and in combination with lisdexamphetamine is provided in Table 1:

| TREATMENT | STATISTICS | 0 | 0.5 | 1 | 1.5 | 2 | 3 | 4 | 6 |
|---|---|---|---|---|---|---|---|---|---|
| SPD503 Alone | N | 40 | 40 | 40 | 40 | 40 | 40 | 40 | 40 |
| | Mean | 0.0026 | 0.3301 | 0.8161 | 1.276 | 1.585 | 1.9714 | 2.2133 | 2.3836 |
| | SD | 0.0165 | 0.2213 | 0.3924 | 0.5644 | 0.7257 | 0.8557 | 0.9627 | 0.9669 |
| | Median | 0 | 0.2729 | 0.7511 | 1.1982 | 1.4905 | 1.694 | 2.1218 | 2.1648 |
| | % CV | 632.5 | 67 | 48.1 | 44.2 | 45.8 | 43.4 | 43.5 | 40.6 |
| | Min | 0 | 0 | 0.3356 | 0.5513 | 0.7099 | 0.9255 | 0.8864 | 0.9106 |
| | Max | 0.1045 | 1.0475 | 2.0569 | 2.8983 | 3.3987 | 4.6963 | 5.1087 | 5.145 |
| SP0503 | N | 41 | 41 | 41 | 41 | 41 | 41 | 41 | 41 |
| Co-administered | Mean | 0 | 0.2679 | 0.6846 | 1.1499 | 1.5008 | 2.0508 | 2.4637 | 2.7105 |
| | SD | 0 | 0.1783 | 0.3221 | 0.4295 | 0.5458 | 0.7247 | 0.856 | 0.8372 |
| | Median | 0 | 0.2439 | 0.6336 | 1.1163 | 1.3472 | 1.9507 | 2.3528 | 2.5718 |
| | % CV | — | 66.6 | 47 | 37.3 | 36.4 | 35.3 | 34.7 | 30.9 |
| | Min | 0 | 0 | 0.1079 | 0.3653 | 0.5614 | 1.0374 | 1.228 | 1.2692 |
| | Max | 0 | 0.7863 | 1.9057 | 2.2846 | 2.6916 | 3.8677 | 4.8733 | 5.23 |

| TREATMENT | STATISTIC | 8 | 12 | 24 | 30 | 48 | 72 |
|---|---|---|---|---|---|---|---|
| SPD503 Alone | | 40 | 40 | 40 | 40 | 40 | 40 |
| | Mean | 2.3247 | 2.0271 | 1.5304 | 1.5296 | 0.9096 | 0.41 |
| | SD | 1.0078 | 0.9012 | 0.6477 | 0.498 | 0.3716 | 0.2548 |
| | Median | 2.0764 | 1.8549 | 1.2854 | 1.5058 | 0.8513 | 0.3555 |
| | % CV | 43.4 | 44.5 | 42.3 | 32.6 | 40.9 | 62.1 |
| | Min | 0.8455 | 0.6883 | 0.603 | 0.7718 | 0.3248 | 0.092 |
| | Max | 5.7862 | 4.7272 | 3.6271 | 2.9928 | 1.8988 | 1.2292 |
| SPD503 | N | 41 | 41 | 41 | 41 | 41 | 41 |
| Co-administered | Mean | 2.7625 | 2.4418 | 1.7413 | 1.5816 | 0.9147 | 0.4214 |
| | SD | 0.9799 | 1.0518 | 0.7261 | 0.5679 | 0.3191 | 0.2435 |
| | Median | 2.4874 | 2.0207 | 1.5996 | 1.5434 | 0.9202 | 0.3512 |
| | % CV | 35.5 | 43.1 | 41.7 | 35.9 | 34.9 | 57.8 |
| | Min | 1.3213 | 1.1148 | 0.7338 | 0 | 0.3492 | 0.1182 |
| | Max | 5.6045 | 5.539 | 3.7705 | 2.8219 | 1.652 | 1.1153 |

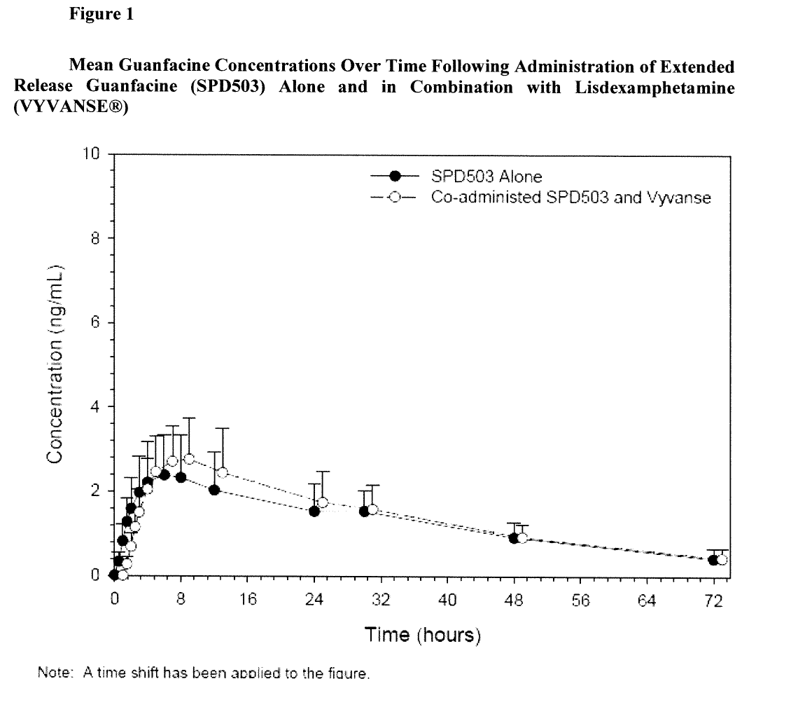

The mean guanfacine plasma concentrations following administration of extended release guanfacine alone and in combination with lisdexamphetamine are shown in FIG. 1. The mean guanfacine plasma concentrations following administration of extended release guanfacine alone were lower than the mean guanfacine plasma concentrations following co-administration with lisdexamphetamine.

A summary of guanfacine pharmacokinetic parameters following administration of extended release guanfacine (SPD503) alone and in combination with lisdexamphetamine (VYVANSE®) is shown in Table 2:

| | Cₘₐₓ | tₘₐₓ | AUC_{0-∞} | t_{1/2} | CL/F | Vz/F |
|---|---|---|---|---|---|---|
| | (ng/mL) | (h) | (ng · h/mL) | (h) | (L/hr/kg) | (L/kg) |
| SPD503 Alone | | | | | | |
| N | 40 | 40 | 37 | 37 | 37 | 37 |
| Mean (SD) | 2.55 (1.03) | 8.6 (7.7) | 104.9 (34.7) | 23.5 (10.2) | 0.54 (0.17) | 17.36 (7.54) |
| Median | 2.30 | 6 | 102.4 | 20.5 | 0.51 | 15.34 |
| Min, Max | 0.98, 5.79 | 1.5, 30 | 54, 218.2 | 11.4, 50 | 0.27, 1.04 | 7.02, 38.05 |
| SPD503 + VYVANSE | | | | | | |
| N | 41 | 41 | 39 | 39 | 39 | 39 |
| Mean (SD) | 2.97 (0.98) | 7.9 (5) | 112.8 (35.7) | 21.4 (8.2) | 0.5 (0.15) | 15.33 (7.35) |
| Median | 2.87 | 6 | 109.4 | 18.8 | 0.46 | 13.61 |
| Min, max | 1.52, 5.60 | 3, 30 | 61.5, 213.6 | 11.9, 48.2 | 0.3, 0.89 | 6.36, 44.79 |

Following oral administration of extended release guanfacine alone, the maximum plasma concentrations were observed at a median of 6 hours after dose administration. The Cₘₐₓ for guanfacine when administered in combination lisdexamphetamine was higher than when administered alone. The guanfacine Cₘₐₓ was outside the standard range for bioequivalence by 7%.

d-Amphetamine Pharmacokinetic Results:

A summary of d-amphetamine plasma concentrations following administration of lisdexamphetamine alone and in combination with extended release guanfacine is shown in Table 3:

| TREATMENT | STATISTICS | 0 | 0.5 | 1 | 1.5 | 2 | 3 | 4 | 6 |
|---|---|---|---|---|---|---|---|---|---|
| d-Amphetamine | N | 41 | 41 | 41 | 41 | 41 | 41 | 41 | 41 |
| Alone | Mean | 0 | 0.1168 | 5.991 | 15.65 | 24.8405 | 33.4712 | 35.3902 | 32.8034 |
| | SD | 0 | 0.5243 | 3.8173 | 6.764 | 8.6231 | 8.3058 | 7.296 | 6.075 |
| | Median | 0 | 0 | 5.32 | 14.99 | 25.77 | 33.64 | 36.91 | 32.26 |
| | % CV | — | 448.8 | 63.7 | 43.2 | 34.7 | 24.8 | 20.6 | 18.5 |
| | Min | 0 | 0 | 0 | 3.29 | 7.39 | 17.11 | 19.61 | 20.51 |
| | Max | 0 | 2.6 | 15.84 | 31.72 | 49.6 | 57.15 | 52.68 | 46.67 |
| d-Amphetamine | N | 41 | 41 | 41 | 41 | 41 | 41 | 41 | 41 |
| Co-administered | Mean | 0 | 0.192 | 5.2517 | 16.08 | 25.4646 | 34.4829 | 35.449 | 32.6424 |
| | SD | 0 | 0.8593 | 3.8871 | 6.871 | 8.3455 | 6.9821 | 5.9684 | 5.1243 |
| | Median | 0 | 0 | 4.6 | 15.83 | 24.83 | 34.23 | 34.87 | 32.21 |
| | % CV | — | 447.7 | 74 | 42.7 | 32.8 | 20.2 | 16.8 | 15.7 |
| | Min | 0 | 0 | 0 | 5.91 | 10.26 | 19.01 | 21.56 | 23.05 |
| | Max | 0 | 4.13 | 20.09 | 31.78 | 43.72 | 53.06 | 48.57 | 44.83 |

| TREATMENT | STATISTICS | 8 | 12 | 24 | 30 | 48 | 72 |
|---|---|---|---|---|---|---|---|
| d-Amphetamine | N | 41 | 41 | 41 | 41 | 41 | 41 |
| Alone | Mean | 29.8083 | 22.869 | 10.7685 | 7.5624 | 2.0566 | 0 |
| | SD | 5.6248 | 5.0822 | 2.9947 | 2.4755 | 1.5259 | 0 |
| | Median | 29.98 | 22.34 | 10.71 | 7.45 | 2.52 | 0 |
| | % CV | 18.9 | 22.2 | 27.8 | 32.7 | 74.2 | — |
| | Min | 17.41 | 10.58 | 4.08 | 2.35 | 0 | 0 |
| | Max | 45.24 | 34.35 | 18.88 | 14.77 | 4.46 | 0 |
| d-Amphetamine | N | 41 | 41 | 41 | 41 | 41 | 41 |
| Co-administered | Mean | 30.422 | 23.7002 | 11.36 | 7.9278 | 1.9466 | 0 |
| | SD | 5.037 | 4.5626 | 2.6637 | 2.2481 | 1.6321 | 0 |
| | Median | 30.61 | 23.82 | 11 | 7.74 | 2.37 | 0 |
| | % CV | 16.6 | 19.3 | 23.4 | 28.4 | 83.8 | — |
| | Min | 20.09 | 15.11 | 6.63 | 3.73 | 0 | 0 |
| | Max | 43.98 | 34.56 | 17.2 | 13.33 | 5.02 | 0 |

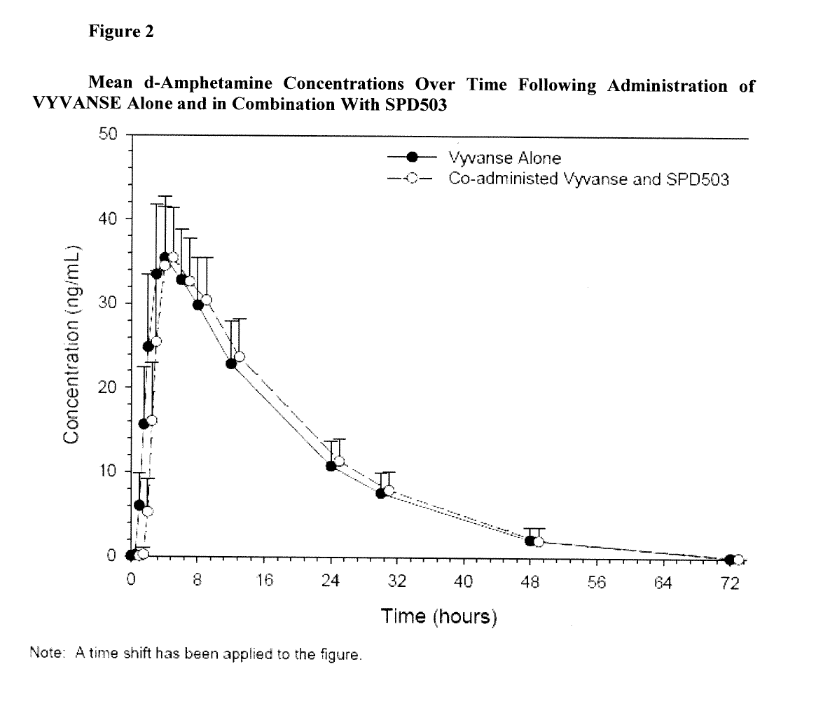

The mean d-amphetamine plasma concentration following administration of lisdexamphetamine alone and in combination with extended release guanfacine is shown in FIG. 2. The mean d-amphetamine plasma concentrations following administration of lisdexamphetamine alone were essentially identical to the mean d-amphetamine plasma concentration following co-administration with extended release guanfacine.

A summary of d-amphetamine pharmacokinetic parameters following the administration of lisdexamphetamine alone and in combination with extended release guanfacine is shown in Table 4:

| | Cmax | trnax | AUC_{0-∞} | t_{1/2} | CL/F | Vz/F |
|---|---|---|---|---|---|---|
| | (ng/mL) | (h) | (ng · h/mL) | (h) | (L/hr/kg) | (L/kg) |
| VYVANSE Alone | | | | | | |
| N | 41 | 41 | 41 | 41 | 41 | 41 |
| Mean (SD) | 36.48 (7.13) | 4.2 (1.1) | 686.9 (159.8) | 11.2 (1.6) | 0.99 (0.23) | 15.58 (2.52) |
| Median | 36.95 | 4 | 687.7 | 11.3 | 0.93 | 15.33 |
| Min, Max | 20.51, 57.15 | 3, 6 | 324.6, 1070 | 8.3, 14.6 | 0.66, 1.8 | 11.16, 21.77 |
| VYVANSE + SPD503 | | | | | | |
| N | 41 | 41 | 41 | 41 | 41 | 41 |
| Mean (SD) | 36.50 (6.00) | 3.9 (1.1) | 708.4 (137.8) | 11.2 (1.5) | 0.95 (0.17) | 15.11 (2.37) |
| Media [symbol] | 35.71 | 4 | 713.6 | 11 | 0.95 | 14.43 |
| Min, max | 23.05, 53.06 | 3, 8 | 456.1, 954.1 | 8, 15.1 | 0.67, 1.34 | 11.45, 23.8 |

Following oral administration of lisdexamphetamine alone, maximum plasma concentrations of d-amphetamine were observed at a median of 4 hours post-dose. Co-administrations of lisdexamphetamine with extended release guanfacine did not alter the pharmacokinetic profile of d-amphetamine. The weight normalized CL/F and weight normalized Vz/F for lisdexamphetamine administered alone and in combination with extended release guanfacine were essentially the same between the two treatments.

Lisdexamphetamine Pharmacokinetic Results:

A summary of lisdexamphetamine plasma concentrations following administration of lisdexamphetamine alone and in combination with extended release guanfacine is shown in Table 5:

| TREATMENT | STATISTIC | 0 | 0.5 | 1 | 1.5 | 2 | 3 | 4 | 6 |
|---|---|---|---|---|---|---|---|---|---|
| | N | 41 | 41 | 41 | 41 | 41. | 41 | 41 | 41 |
| Lisdexamfetamine - | Mean | 0 | 8.5483 | 24.921 | 17.7205 | 9.0166 | 1.8902 | 0.2849 | 0 |
| Alone | SD | 0 | 11.1477 | 15 9087 | 8 9366 | 4.3924 | 1.3336 | 0.5701 | 0 |
| | Median | 0 | 4.51 | 23.56 | 15.72 | 8.17 | 1.87 | 0 | 0 |
| | % CV | — | 130.4 | 63.8 | 50.4 | 48.7 | 70.6 | 200.1 | — |
| | Min | 0 | 0 | 4.5 | 5.87 | 2.42 | 0 | 0 | 0 |
| | Max | 0 | 54.99 | 82.37 | 41.42 | 19.94 | 6.32 | 2.28 | 0 |
| Lisdexamfetamine | N | 41 | 41 | 41 | 41 | 41 | 41 | 41 | 41 |
| Co-administered | Mean | 0 | 8.0107 | 24.199 | 18.969 | 9.6285 | 2.1966 | 0.4885 | 0.0261 |
| | SD | 0 | 16.8709 | 12.4559 | 9.3539 | 6.2476 | 1.7556 | 0.7933 | 0.1671 |
| | Median | 0 | 3.27 | 22.84 | 15.93 | 7.81 | 2.01 | 0 | 0 |
| | % CV | — | 210.6 | 51.5 | 49.3 | 64.9 | 79.9 | 162.4 | 640.3 |
| | Min | 0 | 0 | 3.49 | 7.51 | 2.91 | 0 | 0 | 0 |
| | Max | 0 | 100.85 | 62.26 | 44.67 | 30.3 | 6.93 | 2.76 | 1.07 |

| TREATMENT | STATISTIC | 8 | 12 | 24 | 30 | 48 | 72 |
|---|---|---|---|---|---|---|---|
| Lisdexamfetamine | N | 41 | 41 | 41 | 41 | 41 | 41 |
| Alone | Mean | 0 | 0 | 0 | 0 | 0 | 0 |
| | SD | 0 | 0 | 0 | 0 | 0 | 0 |
| | Median | 0 | 0 | 0 | 0 | 0 | 0 |
| | % CV | — | — | — | — | — | — |
| | Min | 0 | 0 | 0 | 0 | 0 | 0 |
| | Max | 0 | 0 | 0 | 0 | 0 | 0 |
| Lisdexamfetamine | N | 41 | 41 | 41 | 41 | 41 | 41 |
| Co-administered | Mean | 0 | 0 | 0 | 0 | 0 | 0 |
| | SD | 0 | 0 | 0 | 0 | 0 | 0 |
| | Med | 0 | 0 | 0 | 0 | 0 | 0 |
| | % CV | — | — | — | — | — | — |
| | Min | 0 | 0 | 0 | 0 | 0 | 0 |
| | Max | 0 | 0 | 0 | 0 | 0 | 0 |

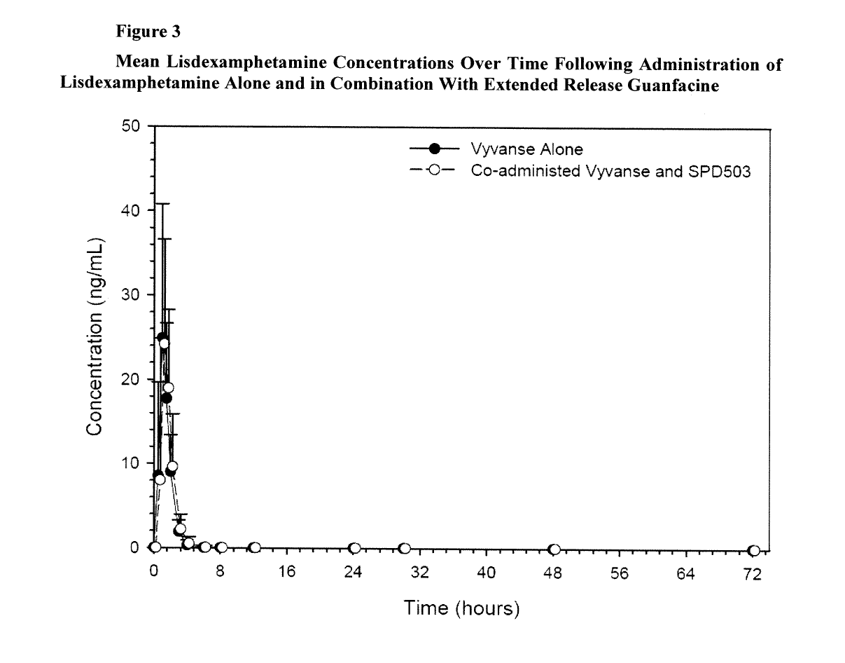

The mean lisdexamphetamine plasma concentration following administration of lisdexamphetamine alone and in combination with extended release guanfacine is shown in FIG. 3. The mean lisdexamphetamine plasma concentrations following administration of lisdexamphetamine alone were essentially identical to the mean lisdexamphetamine plasma concentration following co-administration with extended release guanfacine.

A summary of lisdexamphetamine pharmacokinetic parameters following administration of lisdexamphetamine alone and in combination with extended release guanfacine is shown in Table 6:

| | | | | AUCₗₐₛₜ | AUC_{info} | | | | | |
|---|---|---|---|---|---|---|---|---|---|---|
| | | Cₘₐₓ | Tₘₐₓ | (ng · hr/ | (ng · hr/ | T_{1/2} | CL/F | CL/F | V_{Z}/F | V_{Z}/F |
| TREATMENT | STATISTIC | (ng/mL) | (hr) | mL) | mL) | (hr) | (L/h) | (L/h/kg) | (L) | (L/kg) |
| Lisdexamfetamine | N | 41 | 41 | 41 | 33 | 33 | 33 | 33 | 33 | 33 |
| Alone | Mean | 26.1383 | 1.1 | 32.1 | 36.1 | 0.5 | 1701.28 | 21.71 | 1273.27 | 16.04 |
| | SD | 15.3654 | 0.2 | 18 | 18.9 | 0.1 | 751.79 | 7.84 | 751.62 | 7.8 |
| | Geo Mean | 22.4598 | 1.1 | 28.3 | 32.4 | 0.5 | 1543 | 20.32 | 1094.37 | 14.41 |
| | Median | 25.18 | 1 | 27.2 | 33.2 | 0.5 | 1504.94 | 20.7 | 1035.3 | 13.66 |
| | % CV | 58.8 | 19.3 | 56.2 | 52.2 | 23.9 | 44.2 | 36.1 | 59 | 48.6 |
| | Min | 5.87 | 1 | 10.2 | 14.2 | 0.3 | 522.89 | 6.92 | 288.17 | 3.81 |
| | Max | 82.37 | 1.5 | 94.3 | 95.6 | 0.8 | 3523.94 | 39.64 | 3124.01 | 34.83 |
| Lisdexamfetamine | N | 41 | 41 | 41 | 36 | 36 | 36 | 36 | 36 | 36 |
| Co-administered | Mean | 27.1266 | 1.1 | 33.2 | 37.1 | 0.5 | 1630.59 | 21.2 | 1209.22 | 15.7 |
| | SD | 15.4916 | 0.3 | 19.1 | 19.1 | 0.1 | 647.88 | 7.41 | 510.33 | 5.49 |
| | Geo Mean | 24.0202 | 1.1 | 29 | 33.5 | 0.5 | 1494.75 | 19.8 | 1116.78 | 14.79 |
| | Median | 24.19 | 1 | 29.6 | 31.7 | 0.5 | 1575.19 | 21.11 | 1086.9 | 14.88 |
| | % CV | 57.1 | 24.3 | 57.6 | 51.4 | 27.5 | 39.7 | 34.9 | 42.2 | 35 |
| | Min | 7.88 | 0.5 | 8.2 | 16.6 | 0.3 | 490.31 | 6.49 | 495.23 | 7.06 |
| | Max | 100.85 | 1.5 | 100.4 | 102 | 1 | 3009.62 | 37.98 | 2888.17 | 31.84 |

Lower Limit of Quantification For SPD503 = <0.05 ng/mL. Lower Limit of Quantification for lisdexamfetamine = <1.0 ng/mL: Lower limit of Quantification for d-amphetamine = <2 ng/mL.

Pharmacokinetic Conclusions:

The 90% CI of the geometric mean ratio of guanfacine following extended release guanfacine administration in combination with lisdexamphetamine administration to guanfacine following extended release guanfacine administration alone fell within the interval (0.80, 1.25) for AUC₀₋ₜ and AUC_{0-∞}.

The 90% CI of the geometric mean ratio of guanfacine Cₘₐₓ following extended release guanfacine administration in combination with lisdexamphetamine administration to guanfacine following extended release guanfacine administration alone exceeded the standard interval of bioequivalence by 7%.

The guanfacine Cₘₐₓ was increased by 19% when co-administered with lisdexamphetamine.

The 90% CI of the geometric mean ratio of e-amphetamine following lisdexamphetamine in combination with extended release guanfacine to d-amphetamine alone fell within the interval (0.80, 1.25) for Cₘₐₓ, AUC₀₋ₜ, AUC_{0-∞}.

Adverse Events:

Table 7 is a summary of AEs. No subject had a SAE, a severe AE, or an AE leading to withdrawal from the study.

TABLE 7
Table 13 Summary of Adverse Events

| | Pre- | | Post- | |
|---|---|---|---|---|
| | TEAEs | TEAEs | TEAEs | All AEs |
| Any Events n (%) | 0 (0.0) | 18 (42.9) | 0 (0.0) | 18 (42.9) |
| Serious | 0 (0.0) | 0 (0.0) | 0 (0.0) | 0 (0.0) |
| Severe | 0 (0.0) | 0 (0.0) | 0 (0.0) | 0 (0.0) |
| Leading to withdrawal | 0 (0.0) | 0 (0.0) | 0 (0.0) | 0 (0.0) |
| Related to study drug | 0 (0.0) | 18 (42.9) | 0 (0.0) | 18 (42.9) |

Table 8 shows TEAEs by treatment:

| | SPD503 | VYVANSE | Co- |
|---|---|---|---|
| System Organ Class | 4 mg | 50 mg | Administration |
| Preferred Term | (N = 40) | (N = 41) | (N = 41) |
| Any Event (n %) | 7 (17.5) | 8 (19.5) | 10 (24.4) |
| Cardiac disorders | 0 (0.0) | 3 (7.3) | 2 (4.9) |
| Arrhythmia supraventricular | 0 (0.0) | 1 (2.4) | 1 (2.4) |
| Palpitations | 0 (0.0) | 1 (2.4) | 0 (0.0) |
| Supraventricular extrasystoles | 0 (0.0) | 0 (0.0) | 1 (2.4) |
| Tachycardia | 0 (0.0) | 1 (2.4) | 0 (0.0) |
| Wandering pacemaker | 0 (0.0) | 1 (2.4) | 0 (0.0) |
| Gastrointestinal disorders | 0 (0.0) | 2 (4.9) | 3 (7.3) |
| Diarrhoea | 0 (0.0) | 0 (0.0) | 1 (2.4) |
| Dry mouth | 0 (0.0) | 1 (2.4) | 1 (2.4) |
| Nausea | 0 (0.0) | 1 (2.4) | 1 (2.4) |
| General disorders and | 1 (2.5) | 0 (0.0) | 1 (2.4) |
| administration site conditions | | | |
| Non-cardiac chest pain | 1 (2.5) | 0 (0.0) | 1 (2.4) |
| Nervous system disorders | 7 (17.5) | 4 (9.8) | 7 (17.1) |
| Dizziness | 2 (5.0) | 3 (7.3) | 3 (7.3) |
| Dizziness postural | 4 (10.0) | 1 (2.4) | 0 (0.0) |
| Headache | 3 (7.5) | 2 (4.9) | 3 (7.3) |
| Paraesthesia | 0 (0.0) | 0 (0.0) | 1 (2.4) |
| Psychomotor hyperactivity | 0 (0.0) | 1 (2.4) | 0 (0.0) |
| Psychiatric disorders | 0 (0.0) | 2 (4.9) | 0 (0.0) |
| Nervousness | 0 (0.0) | 2 (4.9) | 0 (0.0) |
| Reproductive system and | 0 (0.0) | 1 (2.4) | 0 (0.0) |
| breast disorders | | | |
| Dysmenorrhoea | 0 (0.0) | 1 (2.4) | 0 (0.0) |
| Vascular disorders | 0 (0.0) | 0 (0.0) | 2 (4.9) |
| Flushing | 0 (0.0) | 0 (0.0) | 1 (2.4) |
| Orthostatic hypotension | 0 (0.0) | 0 (0.0) | 1 (2.4) |

Biochemistry:

There were no clinically meaningful differences in biochemistry results across the treatment groups.

Patents, published patent applications, and non-patent publications cited herein are hereby incorporated by reference in their entirety.

1. A method for treating Attention Deficit Hyperactivity Disorder (ADHD) in a patient which comprises administering to the patient an extended release guanfacine composition adjunctively with a lisdexamphetamine composition.

2. The method of claim 1 wherein the extended release guanfacine composition is administered orally once per day.

3. The method of claim 2 wherein the extended release guanfacine composition dosage is 1 mg, 2 mg, 3 mg, or 4 mg.

4. The method of claim 2 wherein the extended release guanfacine composition dosage is 0.05 mg/kg/day to 0.12 mg/kg/day.

5. The method of claim 1 comprising lisdexamphetamine dimesylate.

6. The method of claim 1 wherein the extended release guanfacine composition is administered in the morning or in the evening.

7. The method of claim 1 wherein the extended release guanfacine composition comprises guanfacine hydrochloride, hypromellose and methacrylic acid copolymer.

8. The method of claim 1 wherein the lisdexamphetamine is administered in a single daily dose of 20 mg, 30 mg, 40 mg, 50 mg, 60 mg, or 70 mg.

9. The method of claim 1 wherein the lisdexamphetamine composition comprises lisdexamfetamine dimesylate, microcrystalline cellulose, croscarmellose sodium, and magnesium stearate.

10. The method of claim 1 wherein the patient is a child.

11. The method of claim 1 wherein the patient is an adolescent.

12. The method of claim 1 wherein the patient is an adult.

13. The method of claim 1 wherein the patient had a suboptimal response to ADHD treatment without an extended release guanfacine composition.

14. The method of claim 1 wherein the treatment is first-line treatment for the patient's ADHD.

15. A method for treating Attention Deficit Hyperactivity Disorder (ADHD) in a patient which comprises administering to the patient a lisdexamphetamine composition adjunctively with an extended release guanfacine composition.
